(19) United States
(12) Patent Application Publication
(10) Pub. No.: US 2022/0265785 A1
(43) Pub. Date: Aug. 25, 2022

(54) COMPOSITION FOR PREVENTING OR TREATING URIC ACID-RELATED DISEASE

(71) Applicants: GUANGZHOU CENTURY CLINICAL RESEARCH CO., LTD, Guangzhou (CN); GUANGZHOU XIN-CHUANGYI BIOPHARMACEUTICAL CO., LTD, Guangzhou (CN); GUANGZHOU WELMAN NEW DRUG R&D CO., LTD., Guangzhou (CN); XIANGBEI WELMAN PHARMACEUTICAL CO., LTD, Changsha, Hunan (CN); NANJING KANGFUSHUN PHARMACEUTICAL CO., LTD, Nanjing, Jiangsu (CN)

(72) Inventors: Mingjie SUN, Guangzhou (CN); Tianyu SUN, Guangzhou (CN); Juanyan JIANG, Guangzhou (CN)

(73) Assignees: GUANGZHOU CENTURY CLINICAL RESEARCH CO., LTD, Guangzhou (CN); GUANGZHOU XIN-CHUANGYI BIOPHARMACEUTICAL CO., LTD, Guangzhou (CN); GUANGZHOU WELMAN NEW DRUG R&D CO., LTD., Guangzhou (CN); XIANGBEI WELMAN PHARMACEUTICAL CO., LTD, Changsha, Hunan (CN); NANJING KANGFUSHUN PHARMACEUTICAL CO., LTD, Nanjing, Jiangsu (CN)

(21) Appl. No.: 17/625,158

(22) PCT Filed: Jul. 17, 2020
(86) PCT No.: PCT/CN2020/102823

§ 371 Date: Jan. 6, 2022

(30) Foreign Application Priority Data
Aug. 29, 2019 (CN) 201910809138.9

(51) Int. Cl. A61K 38/47; A61P 19/06; A61K 9/00

(52) U.S. Cl. CPC A61K 38/47; A61P 19/06; A61K 9/0053

(57) ABSTRACT

Disclosed in the present invention is use of lysozyme in the preparation of a drug for preventing or treating a uric acid-related disease, a drug for reducing uric acid, and a drug for promoting uric acid excretion. Also disclosed in the present invention are a pharmaceutical composition for preventing or treating a uric acid-related disease, a pharmaceutical composition for reducing uric acid, and a pharmaceutical composition for promoting uric acid excretion, and a method for preventing or treating a uric acid-related disease, reducing uric acid and promoting uric acid excretion.

### TECHNICAL FIELD

The present disclosure relates to the field of medicine, in particular to the use of lysozyme in the preparation of a medicament for the preventing or treating a uric acid-related disease, a medicament for reducing uric acid, and a medicament for promoting uric acid excretion. In addition, the present disclosure further relates to a pharmaceutical composition for preventing or treating a uric acid-related disease, a pharmaceutical composition for reducing uric acid, and a pharmaceutical composition for promoting uric acid excretion, and methods for the preventing or treating a uric acid-related disease, reducing uric acid and promoting uric acid excretion.

### BACKGROUND

Uric acid is a metabolite in organisms. The precursors thereof are purine substances, which can be divided into endogenous and exogenous. Endogenous substances include nucleic acids and other purine compounds (such as adenine and guanine) that are decomposed by cell metabolism, and exogenous substances include purines ingested from food. Hypoxanthine and xanthine are direct precursors of uric acid. Under the action of xanthine oxidase, hypoxanthine is oxidized into xanthine, and xanthine is oxidized into uric acid.

Since the human body lacks an enzyme (uric acid oxidase) that further metabolizes uric acid, uric acid is excreted as a final product. About two-thirds of uric acid is excreted through the kidneys via urine, and about one-third of uric acid is excreted through the intestinal tract via feces.

The normal uric acid level in human blood is less than 420 μmol/L for male and less than 360 μmol/L for female. When disorders of purine metabolism or uric acid excretion occur in vivo, the blood uric acid level often exceeds the normal range, presenting pathological conditions such as hyperuricemia. Persistent high uric acid may also cause other metabolic diseases, cardiovascular and cerebrovascular diseases, inflammatory diseases, or substantial organ damage.

The current methods for reducing uric acid are mainly to reduce production of uric acid and increase excretion of uric acid. Existing drugs for reducing production of uric acid mainly include xanthine oxidase inhibitors such as allopurinol and febuxostat, and drugs that increase excretion of uric acid mainly include renal tubular reabsorption inhibitors such as probenecid, benzbromarone, and Lesinurad.

Although existing methods can effectively reduce the level of uric acid, adverse reactions are worrying. Examples include the hepatic and renal toxicity of allopurinol, the cardiotoxicity of febuxostat, the nephrotoxicity of Lesinurad, etc. In addition, people with high uric acid are mostly middle-aged and elderly people whose organ functions are in decline, and these people usually have relatively poor tolerance to adverse reactions. Therefore, the existing methods for reducing uric acid are greatly limited. There is an urgent need to find a safer method for reducing uric acid.

Lysozyme, also called muramidase, is widely found in nature. It has various pharmacological effects such as antibacterial, antifungal and antitumor effects. It also has the characteristics of good biocompatibility, and no irritation and toxicity to tissues. Thus it has been produced as an enzyme preparation with excellent properties in killing pathogenic microorganisms without damaging the body.

Lysozyme plays an important role in non-specific immunity in the human body. It has a variety of biological activities such as bactericidal and anti-inflammatory effects, promoting tissue repair, and enhancing immunity. It is a pharmaceutical enzyme with excellent properties. Therefore, it has been widely used in the field of medicine, e.g., for the treatment of acute and chronic rhinopharyngitis, oral ulcers, chicken pox, herpes zoster, flat wart, etc.

### SUMMARY

An object of the present disclosure is to provide the use of lysozyme in the preparation of a medicament for preventing or treating a uric acid-related disease, a medicament for reducing uric acid, and a medicament for promoting uric acid excretion.

Another object of the present disclosure is to provide a pharmaceutical composition for preventing or treating a uric acid-related disease, a pharmaceutical composition for reducing uric acid, and a pharmaceutical composition for promoting uric acid excretion.

A further object of the present disclosure is to provide methods for the preventing or treating a uric acid-related disease and for reducing uric acid and promoting uric acid excretion.

In particular, the present disclosure relates to the following technical solutions. Provided is the use of lysozyme in the preparation of a medicament for preventing or treating a uric acid-related disease, wherein the uric acid-related disease refers to a disease related to an abnormal uric acid value in an organism.

In some embodiments, the uric acid-related disease is at least one selected from the group consisting of hyperuricemia, uric acid nephropathy, and gout.

In some embodiments, the uric acid-related disease is also accompanied by impaired renal function.

Provided is the use of lysozyme in the preparation of a medicament for reducing uric acid.

In some embodiments, the uric acid is uric acid in the blood of a mammal.

In some embodiments, the mammal is a human being.

In some embodiments, renal function of the mammal is impaired.

Provided is the use of lysozyme in the preparation of a medicament for promoting uric acid excretion.

In some embodiments, the uric acid excretion refers to the excretion of uric acid via the kidneys and/or the intestinal tract.

In some embodiments, the uric acid is uric acid in the blood of a mammal.

In some embodiments, the mammal is a human being.

In some embodiments, renal function of the mammal is impaired.

Provided is a pharmaceutical composition for treating or preventing a uric acid-related disease, wherein the pharmaceutical composition contains lysozyme.

In some embodiments, the uric acid-related disease is at least one selected from the group consisting of hyperuricemia, uric acid nephropathy, and gout.

Provided is a pharmaceutical composition for reducing uric acid, wherein the pharmaceutical composition contains lysozyme.

In some embodiments, the uric acid is uric acid in the blood of a mammal.

In some embodiments, the mammal is a human being.

In some embodiments, renal function of the mammal is impaired.

Provided is a pharmaceutical composition for promoting uric acid excretion, wherein the pharmaceutical composition contains lysozyme.

In some embodiments, the uric acid excretion refers to the excretion of uric acid via the kidneys and/or the intestinal tract.

In some embodiments, the uric acid is uric acid in the blood of a mammal.

In some embodiments, the mammal is a human being.

In some embodiments, renal function of the mammal is impaired.

In some embodiments of the above-mentioned three pharmaceutical compositions, the dosage form of the pharmaceutical composition may be any one of an oral preparation, an injection preparation, and an inhalation preparation; and preferably, the oral preparation is an oral enteric preparation, including but not limited to enteric tablets, enteric capsules, enteric soft capsules, enteric pills, enteric pellets, enteric dripping pills, enteric granules, enteric nanoparticles, enteric sustained-release preparations, and enteric controlled-release preparations.

In some embodiments, the oral enteric preparation is a preparation that releases lysozyme in the large intestine, particularly a preparation that releases lysozyme in the colon.

In some embodiments, the oral enteric preparation is a preparation that releases lysozyme in the small intestine, particularly a preparation that releases lysozyme in the ileum or jejunum.

In some embodiments, the potency of the lysozyme in the pharmaceutical composition is more than 20000 U/mg.

For preparing the above-mentioned pharmaceutical compositions, lysozyme can be used alone, or lysozyme can be prepared into a specific preparation form with a pharmaceutically applicable excipient. The pharmaceutically applicable excipient include, but not limited to, a diluent, a filler, a lubricant, a disintegrant, a glidant, an absorption enhancer, a flavoring agent, a polymeric sustained-release material, an enteric matrix material, a coating material, etc.

Provided is a method of preventing or treating a uric acid-related disease, comprising the step of administering an effective amount of lysozyme to a subject suffering from the uric acid-related disease.

In some embodiments, the uric acid-related disease refers to a disease related to an abnormal uric acid value in an organism.

In some embodiments, the uric acid-related disease is at least one selected from the group consisting of hyperuricemia, uric acid nephropathy, and gout.

In some embodiments, renal function of the subject suffering from the uric acid-related disease is normal.

In some embodiments, renal function of the subject suffering from the uric acid-related disease is impaired.

In some embodiments, the lysozyme is administered through any dosage form of an oral preparation, an injection preparation, and an inhalation preparation; and preferably, the oral preparation is an oral enteric preparation, including but not limited to enteric tablets, enteric capsules, enteric soft capsules, enteric pills, enteric pellets, enteric dripping pills, enteric granules, enteric nanoparticles, enteric sustained-release preparations, and enteric controlled-release preparations.

In some embodiments, the oral enteric preparation is a preparation that releases lysozyme in the large intestine, particularly a preparation that releases lysozyme in the colon.

In some embodiments, the oral enteric preparation is a preparation that releases lysozyme in the small intestine, particularly a preparation that releases lysozyme in the ileum or jejunum.

In some embodiments, the potency of the lysozyme in the oral preparation is more than 20000 U/mg.

In some embodiments, the dosage of the oral preparation is 0.1-20 g/day; preferably, the dosage of the oral preparation is 0.3-15 g/day; and more preferably, the dosage of the oral preparation is 0.5-6 g/day.

In some embodiments, after the dosage of the oral preparation is determined, the oral preparation is administered 1-3 times a day.

Provided is a method of reducing uric acid, comprising the step of administering an effective amount of lysozyme to a subject in need thereof.

In some embodiments, the uric acid is uric acid in the blood of a mammal.

In some embodiments, the mammal is a human being.

In some embodiments, the blood uric acid level of the subject in need thereof is excessively high.

In some embodiments, renal function of the subject in need thereof is normal.

In some embodiments, in addition to the excessively high blood uric acid level, renal function of the subject in need thereof is impaired.

In some embodiments, the lysozyme is administered through any dosage form of an oral preparation, an injection preparation, and an inhalation preparation; and preferably, the oral preparation is an oral enteric preparation, including but not limited to enteric tablets, enteric capsules, enteric soft capsules, enteric pills, enteric pellets, enteric dripping pills, enteric granules, enteric nanoparticles, enteric sustained-release preparations, and enteric controlled-release preparations.

In some embodiments, the oral enteric preparation is a preparation that releases lysozyme in the large intestine, particularly a preparation that releases lysozyme in the colon.

In some embodiments, the oral enteric preparation is a preparation that releases lysozyme in the small intestine, particularly a preparation that releases lysozyme in the ileum or jejunum.

In some embodiments, the potency of the lysozyme in the oral preparation is more than 20000 U/mg.

In some embodiments, the dosage of the oral preparation is 0.1-20 g/day; preferably, the dosage of the oral preparation is 0.3-15 g/day; and more preferably, the dosage of the oral preparation is 0.5-6 g/day.

In some embodiments, after the dosage of the oral preparation is determined, the oral preparation is administered 1-3 times a day.

Provided is a method of promoting uric acid excretion, comprising the step of administering an effective amount of lysozyme to a subject in need thereof.

In some embodiments, the uric acid is uric acid in the blood of a mammal.

In some embodiments, the mammal is a human being.

In some embodiments, the blood uric acid level of the subject in need thereof is excessively high.

In some embodiments, renal function of the subject in need thereof is normal.

In some embodiments, in addition to the excessively high blood uric acid level, renal function of the subject in need thereof is impaired.

In some embodiments, the lysozyme is administered through any dosage form of an oral preparation, an injection preparation, and an inhalation preparation; and preferably, the oral preparation is an oral enteric preparation, including but not limited to enteric tablets, enteric capsules, enteric soft capsules, enteric pills, enteric pellets, enteric dripping pills, enteric granules, enteric nanoparticles, enteric sustained-release preparations, and enteric controlled-release preparations.

In some embodiments, the oral enteric preparation is a preparation that releases lysozyme in the large intestine, particularly a preparation that releases lysozyme in the colon.

In some embodiments, the oral enteric preparation is a preparation that releases lysozyme in the small intestine, particularly a preparation that releases lysozyme in the ileum or jejunum.

In some embodiments, the potency of the lysozyme in the oral preparation is more than 20000 U/mg.

In some embodiments, the dosage of the oral preparation is 0.1-20 g/day; preferably, the dosage of the oral preparation is 0.3-15 g/day; and more preferably, the dosage of the oral preparation is 0.5-6 g/day.

In some embodiments, after the dosage of the oral preparation is determined, the oral preparation is administered 1-3 times a day.

Beneficial Effects of the disclosure:

The methods, medicaments and uses provided by the present disclosure can significantly reduce the blood uric acid level and have high safety. In particular, for people with relatively poor hepatic and renal function, or people with relatively poor tolerance such as infants, pregnant women, and middle-aged and elderly people, the present disclosure has obvious clinical advantages.

### DETAILED DESCRIPTION

Examples are further listed below to illustrate the present disclosure in detail. It should also be understood that the following examples are only used to further illustrate the present disclosure and should not be construed as limiting the scope of the present disclosure, and some non-essential improvements and adjustments made by those skilled in the art based on the principles set forth in the present disclosure all fall within the scope of the present disclosure. The specific process parameters, etc. in the following examples are also only examples in the appropriate ranges, that is, those skilled in the art would have been able to make selections within the appropriate ranges through the description herein, without limitation to the specific data exemplified hereinbelow.

Definition:

The Chinese term Rongjunmei, i.e., lysozyme of the present disclosure, can be lysozyme derived from animals, plants or microorganisms, or a recombinant product of natural lysozyme. For example, it can be egg white lysozyme, human lysozyme, recombinant human lysozyme, phage lysozyme, etc. The lysozyme in the present disclosure includes pharmaceutically acceptable salts thereof, such as hydrochloride, chloride, sulfate, or amino acid salts. Lysozyme was first discovered by Fleming as an endogenous enzyme widely present in organisms. Lysozyme has been approved for edible or medicinal use worldwide. In the United States lysozyme has been Generally Recognized as Safe(GRAS). WHO, many European countries,

Japan and China have approved it to be used as a food additive. It has been approved for medicinal use in China, Japan, Singapore, and other countries for the treatment of rhinitis, laryngopharyngitis, oral ulcers, chickenpox, herpes zoster, flat warts, etc. Products on the market include Neuzym®, Mucozome®, Laisuorui®, etc. The mechanism of the antibacterial effect of lysozyme mainly involves hydrolyzing peptidoglycan in the cell wall of bacteria, and the mechanism of the antiviral effect mainly involves charge interaction with negatively charged viruses.

Enteric preparation: An enteric preparation refers to a preparation that does not release or hardly releases the drug in the stomach but can release most or all of the drug in certain parts of the intestine when entering the intestine. The human intestine includes the small intestine and the large intestine, wherein the small intestine is further divided into duodenum, jejunum, and ileum, and the large intestine is further divided into cecum, colon, and rectum. Different parts of the digestive tract have different pH values. For example, the pH value in the stomach is about 1-3, the pH value in the small intestine is about 4-7, and the pH value in the large intestine is about 7-8. By selecting a pH-dependent degradable material as an excipient, it is possible to prepare a preparation that directionally releases the drug in a specific part of the digestive tract, e.g., a small intestine enteric preparation or a large intestine enteric preparation. In particular, the preparation may include duodenal enteric preparations, jejunal enteric preparations, ileal enteric preparations, cecal enteric preparations, colonic enteric preparations, or rectal enteric preparations, etc.

uric acid-related disease: A uric acid-related disease refers to a disease related to an abnormal uric acid level in an organism, especially a disease related to an excessively high blood uric acid level, including but not limited to hyperuricemia, gout, uric acid nephropathy, and other diseases caused by high uric acid. The normal blood uric acid level is about less than 420 μmol/L for male and about less than 360 μmol/L for female. When the blood uric acid level is higher than the normal range, it is namely a high uric acid state, which can cause a pathological condition or cause a uric acid-related disease.

Impaired renal function: Impaired renal function refers to abnormal renal function. Clinically, renal function can be evaluated by methods such as determining the serum creatinine level, which is well known to those in the art.

The present disclosure will be specifically illustrated below through animal tests.

Note: The test animals, reagents etc. used in the examples were all commercially available. The relevant test methods used in the experiments, the specific operation methods of the relevant instruments, etc., were well known to those skilled in the art.

Lysozyme granules and enteric lysozyme granules were homemade. The preparation method involved: taking lysozyme with a potency of more than 20000 U/mg as a raw material, adding the same amount of starch, mixing them until uniform, adding a binder (water) for granulation, and drying the granules to obtain lysozyme granules. The prepared lysozyme granules were coated with an enteric coating (HPMC) to obtain enteric lysozyme granules.

Benzbromarone fine granules were obtained by grounding commercially available benzbromarone tablets into fine granules.

Example 1 Effect of lysozyme on the uric acid level of hyperuricemia model animals 1.1 Test drug:

Lysozyme granules, enteric lysozyme granules, and benzbromarone fine granules. Each test drug was moistened and uniformly mixed with 0.5% sodium carboxymethylcellulose for easy administration.

1.2 Test animal:

SD rats. The rats had free access to drinking water and food, and they were adaptively reared for one week before test.

1.3 Grouping and administration:

Except for rats in the blank group, the other rats were modeled for 12 days, and the serum uric acid level of each rat was detected. Then the rats were evenly divided into groups according to the uric acid level of each rat, with 8 animals in each group.

Blank group: No drugs were given.

Model control group: 5 ml of 0.5% sodium carboxymethylcellulose was taken orally once a day.

Lysozyme groups and enteric lysozyme groups: High and low doses of the lysozyme granules and enteric lysozyme granules were respectively taken orally, and the dosages were 10 mg/kg and 30 mg/kg based on the active ingredient, once a day.

Benzbromarone group: The benzbromarone fine granules were taken orally at a dose of 30 mg/kg based on the active ingredient, once a day.

1.4 Modeling and test method:

The test was carried out for a total of 18 days.

On day 1 of the test, except for the animals in the blank group, the animals in the other groups were orally given a modeling agent (1.5 g/kg potassium oxonate +0.3 g/kg uric acid, dissolved in 0.5% sodium carboxymethylcellulose) once to start modeling, and thereafter, the same amount of the modeling agent was administrated at the same time every day. 0.5 ml of blood was taken from the orbit before the start of the test (DO) and on day 12

(D12) of the test, respectively, and centrifuged, then the serum was taken and tested for serum uric acid level using a uric acid assay kit and a microplate reader; in addition, 0.5 g of fresh midsection rat feces were taken, diluted and uniformly mixed with four times volume of phosphate buffer, centrifuged, ultrasonically mixed until uniform, incubated at 100° C. for 40 minutes, cooled and then centrifuged, and the supernatant was taken and tested for fecal uric acid level using the uric acid assay kit and the microplate reader.

A significant increase in the serum uric acid levels in the animals indicated that the modeling was successful. Thereafter, the rats were grouped according to the serum uric acid value of each rat. From day 13 (D13), each rat was given the test drug intragastrically every day, as well as the modeling agent every day. The interval between the administration of the test drug and the administration of the modeling agent was more than 1 hour. The drug administration was stopped after day 18 (D18) of the test, and the serum uric acid levels and fecal uric acid levels were determined according to the previous method.

1.5 Test results, analysis and processing:

The collected data, statistical analysis, and main results were shown in Tables 1 and 2.

TABLE 1
Effects of lysozyme on animal serum uric acid levels (X ± SD)

| | Dosage of | | | |
|---|---|---|---|---|
| | administration | Serum uric acid content (μmol/L) | | |
| Group | (mg/kg) | Day 0 | Day 12 | Day 18 |
| Blank group | — | 41.80 ± 3.63 | 44.18 ± 3.96 | 45.02 ± 4.60 |
| Model control group | — | 42.26 ± 3.53 | 118.23 ± 13.31^{##} | 192.23 ± 28.67^{##} |
| Benzbromarone group | 30 | 41.02 ± 3.71 | 117.14 ± 17.74^{##} | 153.45 ± 26.17^{##}** |
| Lysozyme | 10 | 43.66 ± 4.16 | 122.12 ± 10.27^{##} | 161.97 ± 22.11^{##}* |
| group | 30 | 40.49 ± 3.33 | 120.79 ± 17.64^{##} | 149.76 ± 20.12^{##}** |
| Enteric lysozyme group | 10 | 44.30 ± 3.09 | 124.71 ± 19.29^{##} | 137.25 ± 24.51^{##}** |
| | 30 | 46.02 ± 3.80 | 118.01 ± 14.80^{##} | 122.38 ± 20.71^{##}** |

Note:
^{##}p < 0.01, compared with the blank group;
*p < 0.05, compared with the model control group; and
**p < 0.01, compared with the model control group.

TABLE 2
Effects of lysozyme on animal fecal uric acid levels (X ± SD)

| | Dosage of | | | |
|---|---|---|---|---|
| | administration | Fecal uric acid content (mmol/L) | | |
| Group | (mg/kg) | Day 0 | Day 12 | Day 18 |
| Blank group | — | 1.04 ± 0.17 | 1.02 ± 0.23 | 1.21 ± 0.22 |
| Model control group | — | 1.13 ± 0.12 | 1.43 ± 0.22^{##} | 1.59 ± 0.19 |
| Benzbromarone group | 30 | 1.12 ± 0.17 | 1.25 ± 0.31 | 1.34 ± 0.31 |
| Lysozyme group | 10 | 0.98 ± 0.13 | 1.26 ± 0.24 | 1.84 ± 0.20^{##} |
| | 30 | 1.15 ± 0.16 | 1.24 ± 0.20 | 1.98 ± 0.20^{##}* |
| Enteric lysozyme group | 10 | 1.07 ± 0.15 | 1.34 ± 0.26^{#} | 2.07 ± 0.31^{##}** |
| | 30 | 1.09 ± 0.12 | 1.29 ± 0.17 | 2.28 ± 0.38^{##}** |

Note:
^{#}p < 0.05, compared with the blank group;
^{##}p < 0.01, compared with the blank group;
*p < 0.05, compared with the model control group; and
**p < 0.01, compared with the model control group.

In this test, the method of administering exogenous uric acid and a uricase inhibitor resulted in hyperuricemia in rats. Benzbromarone, a drug that promoted uric acid excretion, was used as a positive control drug. The effects of lysozyme on the uric acid levels of the model animals were observed.

The serum uric acid test results showed that the serum uric acid in the model control group was significantly increased on day 12, indicating that the modeling was successful. On day 18, the serum uric acid levels in the lysozyme group and the enteric lysozyme group were significantly lower than those in the model control group, indicating that lysozyme and enteric lysozyme could both significantly reduce the serum uric acid levels in the model animals. In addition, the effect of the enteric lysozyme in reducing serum uric acid was even superior to that of the positive control drug benzbromarone.

The fecal uric acid test results showed that the fecal uric acid level in the model control group was significantly increased on day 12, indicating that probably due to the stimulating effect of the exogenous uric acid, uric acid excretion via the intestinal tract increased in a compensatory manner. On day 18, the fecal uric acid levels in the lysozyme group and the enteric lysozyme group were significantly higher than those in the model control group, whereas the fecal uric acid levels in the positive drug benzbromarone group did not increase significantly, indicating its incapability to promote uric acid excretion via the intestinal tract; while lysozyme, particularly the enteric lysozyme, could significantly promote uric acid excretion via the intestinal tract.

The promotion of the uric acid excretion via the intestinal tract by lysozyme might be one of the reasons for the reduction of the serum uric acid level. There are two ways to excrete uric acid. Most of uric acid is excreted through the kidney, and a small part of uric acid is excreted through the intestinal tract. URAT1 in the renal tubule is an important transporter for the reabsorption of uric acid into the blood. Inhibition of URAT1 can promote the uric acid excretion through the kidney. Transporters such as ABCG2 in intestinal epithelial cells can transport uric acid in the blood to the intestinal tract and promote the excretion of uric acid through the intestine. At present, an overwhelming majority of drugs which reduce uric acid through promoting the excretion, such as benzbromarone, work by promoting renal excretion.

However, for hyperuricemia patients with impaired renal function, the drugs hardly take effect well, in addition they may worsen the renal impairment. In contrast, the drugs which promote uric acid excretion via the intestinal tract can reduce the serum uric acid level without being restricted by renal function, protect renal function, and have better safety, therefore presenting obvious advantages.

The administration of lysozyme enteric tablets (produced by Xiangbei Welman Pharmaceutical Co., Ltd.) by two patients with hyperuricemia is described below to specifically illustrate the present disclosure.

Example 2 Effect of lysozyme enteric tablets on patient with hyperuricemia accompanied by impaired renal function

A 62-year-old female with mild obesity was found to have a serum uric acid value of 550 μmol/L and a serum creatinine value of 192 μmol/L in a physical examination, suggesting moderate hyperuricemia and moderate impaired renal function. Thereafter, she took the lysozyme enteric tablets daily (once in the morning and once in the evening per day, 1 g each time). After two months, a physical examination was performed and it was found that the serum uric acid value decreased to 430 μmol/L, and the serum creatinine value also decreased to 140 μmol/L. After continued use for another two months, the serum uric acid value and the serum creatinine value both returned to normal levels.

Example 3 Effect of lysozyme enteric tablets on patient with hyperuricemia accompanied by gout

A 51-year-old male suffering from gout had obvious redness, swelling and pain in the ankles and toes, and difficulty in walking. Drugs such as ibuprofen and allopurinol had been used in the past to relieve pain and reduce uric acid; however, the effects were not good, and the serum uric acid value basically remained at a high level of 700 μmol/L or more. The lysozyme enteric tablets were tried daily (once in the morning, once at noon and once in the evening, 2 g each time). One month later, he felt that the frequency of pain episodes decreased and the difficulty in walking was alleviated. After taking lysozyme enteric tablets for a total of four months, the serum uric acid value decreased to 580 μmol/L in an examination. After continued use for another three months, the serum uric acid value returned to the normal level, i.e., 400 μmol/L or lower, the redness and swelling were significantly alleviated, and the pain basically no longer occurred.

The present disclosure has been described in detail above with general descriptions and specific embodiments. Based on the present disclosure, those skilled in the art can easily make some modifications or improvements. Therefore, such modifications or improvements made without departing from the spirit of the present disclosure all fall within the claimed scope of the present disclosure.

1-19. (canceled)

20. A method of preventing or treating a uric acid-related disease, comprising a step of administering an effective amount of lysozyme to a subject suffering from the uric acid-related disease.

21. The method according to claim 20, wherein the uric acid-related disease refers to a disease related to an abnormal uric acid value in an organism.

22. The method according to claim 20, wherein renal function of the subject suffering from the uric acid-related disease is normal or impaired.

23. The method according to claim 20, wherein lysozyme is administered through any dosage form of an oral preparation, an injection preparation, and an inhalation preparation.

24. The method according to claim 23, wherein preparation is an oral enteric preparation, and the oral enteric preparation is a preparation that releases lysozyme in the colon, ileum, or jejunum.

25. A method of reducing uric acid or promoting uric acid excretion, comprising a step of administering an effective amount of lysozyme to a subject in need of reducing uric acid or promoting uric acid excretion.

26. The method according to claim 25, wherein the uric acid is uric acid in the blood of a mammal.

27. The method according to claim 25, wherein the blood uric acid level of the subject is excessively high.

28. The method according to claim 25, wherein lysozyme is administered through any dosage form of an oral preparation, an injection preparation, and an inhalation preparation.

29. The method according to claim 28, wherein the oral preparation is an oral enteric preparation, and the oral enteric preparation is a preparation that releases lysozyme in the colon, ileum, or jejunum.

30. The method according to claim 20, wherein the uric acid-related disease is at least one selected from the group consisting of hyperuricemia, uric acid nephropathy, and gout.

31. The method according to claim 23, wherein the oral preparation is an oral enteric preparation, including but not limited to enteric tablets, enteric capsules, enteric soft capsules, enteric pills, enteric pellets, enteric dripping pills, enteric granules, enteric nanoparticles, enteric sustained-release preparations, and enteric controlled-release preparations.

32. The method according to claim 23, wherein the potency of lysozyme in the oral preparation is more than 20000 U/mg.

33. The method according to claim 23, wherein the dosage of the oral preparation is 0.1-20 g/day.

34. The method according to claim 25, wherein the excretion refers to excretion via the kidney and/or the intestinal tract.

35. The method according to claim 26, wherein the mammal is a human being.

36. The method according to claim 25, wherein renal function of the subject is normal or impaired.

37. The method according to claim 28, wherein the oral preparation is an oral enteric preparation, including but not limited to enteric tablets, enteric capsules, enteric soft capsules, enteric pills, enteric pellets, enteric dripping pills, enteric granules, enteric nanoparticles, enteric sustained-release preparations, and enteric controlled-release preparations.

38. The method according to claim 28, wherein the potency of lysozyme in the oral preparation is more than 20000 U/mg.

39. The method according to claim 28, wherein the dosage of the oral preparation is 0.1-20 g/day.
